(19) United States
(12) Patent Application Publication
(10) Pub. No.: US 2008/0207658 A1
(43) Pub. Date: Aug. 28, 2008

(54) Pharmaceutical Combination of Bcr-Abl and Raf Inhibitors

(76) Inventor: Paul W. Manley, Arlesheim (CH)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 11/909,013

(22) PCT Filed: Feb. 24, 2006
(86) PCT No.: PCT/EP2006/001740

§ 371 Date: Sep. 18, 2007

Related U.S. Application Data
(60) Provisional application No. 60/656,340, filed on Feb. 25, 2005.

(51) Int. Cl. A61K 31/506; A61P 35/00

(52) U.S. Cl. 514/275

(57) ABSTRACT

The invention provides a pharmaceutical combination comprising: a) a pyrimidylaminobenzamide compound, and b) a RAF kinase inhibitor and a method for treating or preventing a proliferative disease using such a combination, wherein compound a) has the following general Formula: (I).

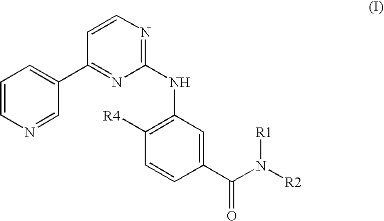

The present invention relates to a pharmaceutical combination comprising a pyrimidylaminobenzamide compound and a RAF inhibitor, and the uses of such a combination e.g. in proliferative diseases, e.g. tumors, myelomas, leukemias, psoriasis, restenosis, sclerodermitis and fibrosis.

In spite of numerous treatment options for proliferative disease patients, there remains a need for effective and safe antiproliferative agents and a need for their preferential use in combination therapy.

### SUMMARY OF THE INVENTION

It has now been found that a combination comprising at least one pyrimidylaminobenzamide compound and a RAF kinase inhibitor, e.g. as defined below, has a beneficial effect on proliferative diseases, e.g. tumors, myelomas, leukemias, psoriasis, restenosis, sclerodermitis and fibrosis.

### DETAILED DESCRIPTION OF THE INVENTION

The present invention relates to the use of pyrimidylaminobenzamide compounds of formula I:

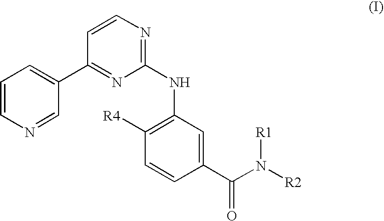

wherein
R₁ represents hydrogen, lower alkyl, lower alkoxy-lower alkyl, acyloxy-lower alkyl, carboxy-lower alkyl, lower alkoxycarbonyl-lower alkyl, or phenyl-lower alkyl;
R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted;
and R₃ represents hydroxy, lower alkoxy, acyloxy, carboxy, lower alkoxycarbonyl, carbamoyl, N-mono- or N,N-disubstituted carbamoyl, amino, mono- or disubstituted amino, cycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted;
or wherein R₁ and R₂ together represent alkylene with four, five or six carbon atoms optionally mono- or disubstituted by lower alkyl, cycloalkyl, heterocyclyl, phenyl, hydroxy, lower alkoxy, amino, mono- or disubstituted amino, oxo, pyridyl, pyrazinyl or pyrimidinyl; benzalkylene with four or five carbon atoms; oxaalkylene with one oxygen and three or four carbon atoms; or azaalkylene with one nitrogen and three or four carbon atoms wherein nitrogen is unsubstituted or substituted by lower alkyl, phenyl-lower alkyl, lower alkoxycarbonyl-lower alkyl, carboxy-lower alkyl, carbamoyl-lower alkyl, N-mono- or N,N-disubstituted carbamoyl-lower alkyl, cycloalkyl, lower alkoxycarbonyl, carboxy, phenyl, substituted phenyl, pyridinyl, pyrimidinyl, or pyrazinyl;
R₄ represents hydrogen, lower alkyl, or halogen;
and a N-oxide or a pharmaceutically acceptable salt of such a compound for the preparation of a pharmaceutical composition for the treatment of kinase dependent diseases.

The general terms used hereinbefore and hereinafter preferably have within the context of this disclosure the following meanings, unless otherwise indicated:

The prefix “lower” denotes a radical having up to and including a maximum of 7, especially up to and including a maximum of 4 carbon atoms, the radicals in question being either linear or branched with single or multiple branching.

Where the plural form is used for compounds, salts, and the like, this is taken to mean also a single compound, salt, or the like.

Any asymmetric carbon atoms may be present in the (R)-, (S)- or (R,S)-configuration, preferably in the (R)- or (S)-configuration. The compounds may thus be present as mixtures of isomers or as pure isomers, preferably as enantiormer-pure diastereomers.

The invention relates also to possible tautomers of the compounds of formula I.

Lower alkyl is preferably alkyl with from and including 1 up to and including 7, preferably from and including 1 to and including 4, and is linear or branched; preferably, lower alkyl is butyl, such as n-butyl, sec-butyl, isobutyl, tert-butyl, propyl, such as n-propyl or isopropyl, ethyl or methyl. Preferably lower alkyl is methyl, propyl or tert-butyl.

Lower acyl is preferably formyl or lower alkylcarbonyl, in particular acetyl.

An aryl group is an aromatic radical which is bound to the molecule via a bond located at an aromatic ring carbon atom of the radical. In a preferred embodiment, aryl is an aromatic radical having 6 to 14 carbon atoms, especially phenyl, naphthyl, tetrahydronaphthyl, fluorenyl or phenanthrenyl, and is unsubstituted or substituted by one or more, preferably up to three, especially one or two substituents, especially selected from amino, mono- or disubstituted amino, halogen, lower alkyl, substituted lower alkyl, lower alkenyl, lower alkynyl, phenyl, hydroxy, etherified or esterified hydroxy, nitro, cyano, carboxy, esterified carboxy, alkanoyl, benzoyl, carbamoyl, N-mono- or N,N-disubstituted carbamoyl, amidino, guanidino, ureido, mercapto, sulfo, lower alkylthio, phenylthio, phenyl-lower alkylthio, lower alkylphenylthio, lower alkylsulfinyl, phenylsulfinyl, phenyl-lower alkylsulfinyl, lower alkylphenylsulfinyl, lower alkylsulfonyl, phenylsulfonyl, phenyl-lower alkylsulfonyl, lower alkylphenylsulfonyl, halogen-lower alkylmercapto, halogen-lower alkylsulfonyl, such as especially trifluoromethanesulfonyl, dihydroxybora (—B(OH)₂), heterocyclyl, a mono- or bicyclic heteroaryl group and lower alkylene dioxy bound at adjacent C-atoms of the ring, such as methylene dioxy. Aryl is more preferably phenyl, naphthyl or tetrahydronaphthyl, which in each case is either unsubstituted or independently substituted by one or two substituents selected from the group comprising halogen, especially fluorine, chlorine, or bromine; hydroxy; hydroxy etherified by lower alkyl, e.g. by methyl, by halogen-lower alkyl, e.g. trifluoromethyl, or by phenyl; lower alkylene dioxy bound to two adjacent C-atoms, e.g. methylenedioxy, lower alkyl, e.g. methyl or propyl; halogen-lower alkyl, e.g. trifluoromethyl; hydroxy-lower alkyl, e.g. hydroxymethyl or 2-hydroxy-2-propyl; lower alkoxy-lower alkyl; e.g. methoxymethyl or 2-methoxyethyl; lower alkoxycarbonyl-lower alkyl, e.g. methoxy-carbonylmethyl; lower alkynyl, such as 1-propynyl; esterified carboxy, especially lower alkoxycarbonyl, e.g. methoxycarbonyl, n-propoxy carbonyl or iso-propoxy carbonyl; N-mono-substituted carbamoyl, in particular carbamoyl monosubstituted by lower alkyl, e.g. methyl, n-propyl or iso-propyl; amino; lower alkylamino, e.g. methylamino; di-lower alkylamino, e.g. dimethylamino or diethylamino; lower alkylene-amino, e.g. pyrrolidino or piperidino; lower oxaalkylene-amino, e.g. morpholino, lower azaalkylene-amino, e.g. piperazino, acylamino, e.g. acetylamino or benzoylamino; lower alkylsulfonyl, e.g. methylsulfonyl; sulfamoyl; or phenylsulfonyl.

A cycloalkyl group is preferably cyclopropyl, cyclopentyl, cyclohexyl or cycloheptyl, and may be unsubstituted or substituted by one or more, especially one or two, substitutents selected from the group defined above as substitutents for aryl, most preferably by lower alkyl, such as methyl, lower alkoxy, such as methoxy or ethoxy, or hydroxy, and further by oxo or fused to a benzo ring, such as in benzcyclopentyl or benzcyclohexyl.

Substituted alkyl is alkyl as last defined, especially lower alkyl, preferably methyl; where one or more, especially up to three, substituents may be present, primarily from the group selected from halogen, especially fluorine, amino, N-lower alkylamino, N,N-di-lower alkylamino, N-lower alkanoylamino, hydroxy, cyano, carboxy, lower alkoxycarbonyl, and phenyl-lower alkoxycarbonyl. Trifluoromethyl is especially preferred.

Mono- or disubstituted amino is especially amino substituted by one or two radicals selected independently of one another from lower alkyl, such as methyl; hydroxy-lower alkyl, such as 2-hydroxyethyl; lower alkoxy lower alkyl, such as methoxy ethyl; phenyl-lower alkyl, such as benzyl or 2-phenylethyl; lower alkanoyl, such as acetyl; benzoyl; substituted benzoyl, wherein the phenyl radical is especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl, and carbamoyl; and phenyl-lower alkoxycarbonyl, wherein the phenyl radical is unsubstituted or especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl, and carbamoyl; and is preferably N-lower alkylamino, such as N-methylamino, hydroxy-lower alkylamino, such as 2-hydroxyethylamino or 2-hydroxypropyl, lower alkoxy lower alkyl, such as methoxy ethyl, phenyl-lower alkylamino, such as benzylamino, N,N-di-lower alkylamino, N-phenyl-lower alkyl-N-lower alkylamino, N,N-di-lower alkylphenylamino, lower alkanoylamino, such as acetylamino, or a substituent selected from the group comprising benzoylamino and phenyl-lower alkoxycarbonylamino, wherein the phenyl radical in each case is unsubstituted or especially substituted by nitro or amino, or also by halogen, amino, N-lower alkylamino, N,N-di-lower alkylamino, hydroxy, cyano, carboxy, lower alkoxycarbonyl, lower alkanoyl, carbamoyl or aminocarbonylamino. Disubstituted amino is also lower alkylene-amino, e.g. pyrrolidino, 2-oxopyrrolidino or piperidino; lower oxaalkylene-amino, e.g. morpholino, or lower azaalkylene-amino, e.g. piperazino or N-substituted piperazino, such as N-methylpiperazino or N-methoxycarbonylpiperazino.

Halogen is especially fluorine, chlorine, bromine, or iodine, especially fluorine, chlorine, or bromine.

Etherified hydroxy is especially C₈-C₂₀alkyloxy, such as n-decyloxy, lower alkoxy (preferred), such as methoxy, ethoxy, isopropyloxy, or tert-butyloxy, phenyl-lower alkoxy, such as benzyloxy, phenyloxy, halogen-lower alkoxy, such as trifluoromethoxy, 2,2,2-trifluoroethoxy or 1,1,2,2-tetrafluoroethoxy, or lower alkoxy which is substituted by mono- or bicyclic hetero-aryl comprising one or two nitrogen atoms, preferably lower alkoxy which is substituted by imidazolyl, such as 1H-imidazol-1-yl, pyrrolyl, benzimidazolyl, such as 1-benzimidazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, indolyl or thiazolyl.

Esterified hydroxy is especially lower alkanoyloxy, benzoyloxy, lower alkoxycarbonyloxy, such as tert-butoxycarbonyloxy, or phenyl-lower alkoxycarbonyloxy, such as benzyloxycarbony loxy.

Esterified carboxy is especially lower alkoxycarbonyl, such as tert-butoxycarbonyl, iso-propoxycarbonyl, methoxycarbonyl or ethoxycarbonyl, phenyl-lower alkoxycarbonyl, or phenyloxycarbonyl.

Alkanoyl is primarily alkylcarbonyl, especially lower alkanoyl, e.g. acetyl.

N-Mono- or N,N-disubstituted carbamoyl is especially substituted by one or two substituents independently selected from lower alkyl, phenyl-lower alkyl and hydroxy-lower alkyl, or lower alkylene, oxa-lower alkylene or aza-lower alkylene optionally substituted at the terminal nitrogen atom.

A mono- or bicyclic heteroaryl group comprising zero, one, two or three ring nitrogen atoms and zero or one oxygen atom and zero or one sulfur atom, which groups in each case are unsubstituted or mono- or polysubstituted, refers to a heterocyclic moiety that is unsaturated in the ring binding the heteroaryl radical to the rest of the molecule in formula I and is preferably a ring, where in the binding ring, but optionally also in any annealed ring, at least one carbon atom is replaced by a heteroatom selected from the group consisting of nitrogen, oxygen and sulfur; where the binding ring preferably has 5 to 12, more preferably 5 or 6 ring atoms; and which may be unsubstituted or substituted by one or more, especially one or two, substituents selected from the group defined above as substituents for aryl, most preferably by lower alkyl, such as methyl, lower alkoxy, such as methoxy or ethoxy, or hydroxy. Preferably the mono- or bicyclic heteroaryl group is selected from 2H-pyrrolyl, pyrrolyl, imidazolyl, benzimidazolyl, pyrazolyl, indazolyl, purinyl, pyridyl, pyrazinyl, pyrimidinyl, pyridazinyl, 4H-quinolizinyl, isoquinolyl, quinolyl, phthalazinyl, naphthyridinyl, quinoxalyl, quinazolinyl, quinnolinyl, pteridinyl, indolizinyl, 3H-indolyl, indolyl, isoindolyl, oxazolyl, isoxazolyl, thiazolyl, isothiazolyl, triazolyl, tetrazolyl, furazanyl, benzo[d]pyrazolyl, thienyl and furanyl. More preferably the mono- or bicyclic heteroaryl group is selected from the group consisting of pyrrolyl, imidazolyl, such as 1H-imidazol-1-yl, benzimidazolyl, such as 1-benzimidazolyl, indazolyl, especially 5-indazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, especially 4- or 8-quinolinyl, indolyl, especially 3-indolyl, thiazolyl, benzo[d]pyrazolyl, thienyl, and furanyl. In one preferred embodiment of the invention the pyridyl radical is substituted by hydroxy in ortho position to the nitrogen atom and hence exists at least partially in the form of the corresponding tautomer which is pyridin-(1H)-2-one. In another preferred embodiment, the pyrimidinyl radical is substituted by hydroxy both in position 2 and 4 and hence exists in several tautomeric forms, e.g. as pyrimidine-(1H, 3H)2,4-dione.

Heterocyclyl is especially a five, six or seven-membered heterocyclic system with one or two heteroatoms selected from the group comprising nitrogen, oxygen, and sulfur, which may be unsaturated or wholly or partly saturated, and is unsubstituted or substituted especially by lower alkyl, such as methyl, phenyl-lower alkyl, such as benzyl, oxo, or heteroaryl, such as 2-piperazinyl; heterocyclyl is especially 2- or 3-pyrrolidinyl, 2-oxo-5-pyrrolidinyl, piperidinyl, N-benzyl-4-piperidinyl, N-lower alkyl-4-piperidinyl, N-lower alkyl-piperazinyl, morpholinyl, e.g. 2- or 3-morpholinyl, 2-oxo-1H-azepin-3-yl, 2-tetrahydrofuranyl, or 2-methyl-1,3-dioxolan-2-yl.

Salts are especially the pharmaceutically acceptable salts of compounds of formula I.

Such salts are formed, for example, as acid addition salts, preferably with organic or inorganic acids, from compounds of formula I with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, a compound of formula I may also form internal salts.

For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

In view of the close relationship between the novel compounds in free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient.

Compounds within the scope of formula I and the process for their manufacture are disclosed in WO 04/005281 published on Jan. 15, 2004 which is hereby incorporated into the present application by reference. A preferred compound is 4-Methyl-3-[[4-(3-pyridinyl)-2-pyrimidinyl]amino]-N-[5-(4-methyl-1H-imidazol-1-yl)-3-(trifluorom ethyl)phenyl]benzamide.

Combination of the present invention include compounds that inhibit RAF kinase, a serine/threonine kinase that functions in the MAP kinase signaling pathway, and to the use of such combinations for the treatment of diseases characterized by an aberrant MAP kinase signaling pathway, e.g., proliferative diseases like certain cancers.

RAF inhibitors are e.g. compounds which inhibit wild-type C-Raf at an IC₅₀ of from 0.05 mmol/L to more than 4.0 mmol/L and/or mutant B-Raf (V599E) at an IC₅₀ of from 0.08 mmol/L to more than 4.0 mmol/L in the following assays:

Test for activity against the RAF kinase: Active B-Raf, C-Raf, and V599E B-Raf proteins of human sequence are purified from insect cells using the baculoviral expression system. Raf inhibition is tested in 96-well microplates coated with IκB-A and blocked with Superblock. The phosphorylation of IκB-α at Serine 36 is detected using a phospho-IκB-α specific antibody (Cell Signaling #9246), an anti-mouse IgG alkaline phosphatase conjugated secondary antibody (Pierce #31320), and an alkaline phosphatase substrate, ATTOPHOS (Promega, #S101).

Suitable RAF inhibitors include e.g.:
Compounds as disclosed in WO 00/09495 which published Feb. 24, 2000, e.g compounds of formula II:

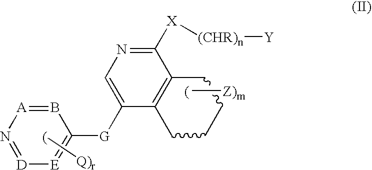

wherein
r is from 0 to 2;
n is from 0 to 2;
m is from 0 to 4;
A, B, D and E are each independently of the others N or CH, with the proviso that not more than two of those radicals are N;
G is lower alkylene, —CH₂—O—, —CH₂—S—, —CH₂—NH—, oxa (—O—), thia (—S—) or imino (—NH—), or is lower alkylene substituted by acyloxy or by hydroxy;
Q is lower alkyl, especially methyl;
R is H or lower alkyl;
X is imino, oxa or thia;
Y is lower alkyl or, especially, aryl, heteroaryl or unsubstituted or substituted cycloalkyl; and
Z is amino, mono- or di-substituted amino, halogen, alkyl, substituted alkyl, hydroxy, etheri-fied or esterified hydroxy, nitro, cyano, carboxy, esterified carboxy, alkanoyl, carbamoyl, N-mono- or N,N-di-substituted carbamoyl, amidino, guanidino, mercapto, sulfo, phenylthio, phenyl-lower alkylthio, alkylphenylthio, phenylsulfinyl, phenyl-lower alkylsulfinyl, alkyl-phenylsulfinyl, phenylsulfonyl, phenyl-lower alkanesulfonyl or alkylphenylsulfonyl, and where, if more than one radical Z is present (m≧2), the substituents Z are identical or different;
and wherein the bonds indicated by a wavy line are either single bonds or double bonds;
or an N-oxide of the mentioned compound, wherein one or more N a toms carry an oxygen atom; or a salt thereof.

Especially preferred is (4-tert-butyl-phenyl)-(4-pyridin-4-ylmethyl-isoquinolin-1-yl)-amine.
Additional RAF inhibitors include compounds selected from [4,7′]biisoquinolinyl-1-yl-4-(tert-butyl-phenyl)-amine, (4-tert-Butyl-phenyl)-(4-quinazolin-6-yl-isoquinolin-1-yl)-amine and[4,7′]Biisoquinolinyl-1-yl-(2-tert-butyl-pyrimidin-5-yl)-amine.
Additional RAF inhibitors include compound as disclosed in WO 04/110452 which published Dec. 23, 2004, e.g compounds of formula III:

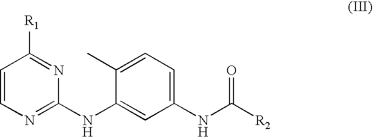

wherein
R₁ is a phenyl radical or a heteroaryl radical; and
R₂ is a phenyl radical;
or an N-oxide or a pharmaceutically acceptable salt thereof.

With respect to compound III, the term “lower” when referring to substituents such as alkyl, alkoxy, alkyl amine, alkylthio and the like denotes a radical having up to and including a maximum of 7, especially from 1 up to and including a maximum of 4, carbon atoms, the radicals in question being unbranched or branched one or more times.

With respect to compound III, the alkyl portion of lower alkyl, lower alkoxy, mono- or di-lower alkyl amino, lower alkyl thio and other substituents with an alkyl portion is especially C₁-C₄alkyl, for example n-butyl, sec-butyl, tert-butyl, n-propyl, isopropyl, methyl or ethyl. Such alkyl substituents are unsubstituted or substituted by halogen, hydroxy, nitro, cyano, lower alkoxy, C₃-C₇ cycloalkyl, amino, or mono- or di-lower alkyl amino, unless otherwise indicated.

With respect to compound II, halo-lower alkyl, halo-lower alkyloxy, halo-lower alkylthio and the like refer to substituents having an alkyl portion wherein the alkyl portion is mono- to completely substituted by halogen. Halo-lower alkyl, halo-lower alkyloxy, halo-lower alkylthio and the like are included within substituted lower alkyl, substituted lower alkoxy, substituted lower alkylthio and the like.

With respect to compound III, halogen is especially fluorine, chlorine, bromine or iodine, more especially fluorine, chlorine or bromine, in particular fluorine.

With respect to compound III, a phenyl radical is generally an unsubstituted phenyl or phenyl that is substituted with from 1-5, preferably 1 or 2, substituents. Appropriate substituents include, but are not limited to, amino, mono- or di-lower alkyl substituted amino, wherein the lower alkyl substituents may be unsubstituted or further substituted by those substituents listed above for alkyl groups, halogen, lower alkyl, substituted lower alkyl, hydroxy, lower alkoxy, substituted lower alkoxy, nitro, cyano, mercapto, lower alkylthio, halo-lower alkylthio, heterocyclyl, heteroaryl, heterocyclylalkyl, heteroarylalkyl, lower alkanoyl, carbamoyl, and N-mono- or N,N-di-lower alkyl substituted carbamoyl, wherein the lower alkyl substituents may be unsubstituted or further substituted.

In Compound III, R₁ as a phenyl radical is especially unsubstituted phenyl or phenyl which is substituted by one or more substituents, preferably up to three, especially one substituent. Especially important substituents for R₁ phenyl radicals include amino, mono- or di-lower alkyl amino, where the alkyl groups are unsubstituted or substituted, halogen, lower alkyl, substituted lower alkyl, hydroxy, lower alkoxy, substituted lower alkoxy, nitro, cyano, mercapto, lower alkylthio and substituted lower alkylthio.

R₁ as a phenyl radical is especially phenyl that is unsubstituted or substituted by one or two identical or different substituents selected from halogen, especially fluorine or chlorine; lower alkyl, especially methyl, ethyl, propyl or t-butyl; halo-lower alkyl, especially trifluoromethyl; hydroxy; lower alkoxy, especially methoxy or ethoxy; halo-lower alkyloxy, such as trifluoromethoxy or 1,1,2,2-tetrafluoroethyloxy; more especially by one substituent selected from unsubstituted or substituted lower alkyl, especially methyl, halo-lower alkyl, such as trifluoromethyl, unsubstituted or substituted lower alkoxy, especially methoxy and halo-lower alkoxy, especially trifluoromethoxy.

With respect to compound III, R₂ as a phenyl radical is generally a phenyl ring that is unsubstituted or preferably substituted by one or more substituents, preferably up to three, especially one or two substituents. The substituents are especially amino, mono- or di-lower alkyl-substituted amino, wherein the alkyl groups are unsubstituted or further substituted, especially by halogen or lower alkoxy; halogen, unsubstituted or substituted lower alkyl, unsubstituted or substituted lower alkoxy, hydroxy, nitro, cyano, lower alkanoyl, carbamoyl, N-mono- or N,N-di-lower alkyl substituted carbamoyl, mercapto, lower alkylthio and halo-lower alkylthio.

R₂ as a phenyl radical is preferably phenyl that is substituted by one or two identical or different substituents selected from halogen, especially fluorine or chlorine; mono- or di-lower alkyl-substituted amino, especially dimethylamino or diethylamino; lower alkyl, especially methyl or ethyl; halo-lower alkyl, especially difluoromethyl, trifluoromethyl, 2,2,2-trifluoroethyl, or 1,1,2,2-tetrafluoroethyl; lower alkoxy, especially methoxy or ethoxy; halo-lower alkoxy, especially difluoromethoxy, trifluoromethoxy, 2,2,2-trifluoroethoxy, or 1,1,2,2-tetrafluoroethoxy, lower alkylthio, such as methylmercapto, halo-lower alkylthio, such as difluoromethylthio, trifluoromethylthio, 2,2,2-trifluoroethylthio, or 1,1,2,2-tetrafluoroethylthio. R₂ as a phenyl radical is more especially phenyl substituted by one or two identical or different substituents selected from unsubstituted or halo-substituted lower alkyl, unsubstituted or halo-substituted lower alkoxy, unsubstituted or halo-substituted lower alkylthio and halogen, especially fluorine or chlorine.

With respect to compound III, a heteroaryl radical is especially a 5 to 7 membered aromatic ring comprising from 1 to 3 heteroatoms selected from N, O and S. The heteroaryl radical is unsubstituted or substituted by one or more, especially from one to three, for example one, identical or different substituents. Important substituents on heteroaryl radicals are those selected from the group consisting of halogen, for example, fluorine or chlorine; mono- or di-lower alkyl-substituted amino wherein the alkyl groups are unsubstituted or substituted by halogen, hydroxy, nitro, cyano, lower alkoxy, C₃-C₇ cycloalkyl, a heterocyclic radical or a heteroaryl radical; lower alkyl, such as methyl or ethyl; halo-lower alkyl, such as trifluoromethyl; lower alkoxy, such as methoxy or ethoxy; halo-lower alkoxy, for example, trifluoromethoxy; lower alkylthio, such as methylmercapto, halo-lower alkylthio, such as trifluoromethylthio, a heteroaryl radical, heteroaryl-lower-alkylene, a heterocyclic radical or heterocyclic-lower-alkylene.

Heteroaryl-lower-alkylene and heterocyclic-lower-alkylene are substituents of the formula het-C₁-C₄-alkylene- where het is a heteroaryl or heterocyclic radical.
Additional RAF inhibitors include compounds as disclosed in WO 04/080464 published Sep. 23, 2004, e.g compounds of formula IV:

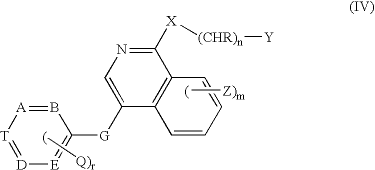

wherein
r is from 0 to 2;
n is from 0 to 2;
m is from 0 to 4;
A, B, D, E and T are each independently of the others N or CH, with the proviso that at least one, but not more than three, of A, B, D, E and T are N;
G is lower alkylene, —CH₂—O—, —CH₂—S—, —CH₂—NH—, —SO₂—, oxa (—O—), thia (—S—) or —NR—, or is lower alkylene substituted by acyloxy, oxo, halogen or hydroxy.
Q is lower alkyl, especially methyl;
R is H or lower alkyl;
X is Y, —N(R)—, oxa or thio; preferably —NH—;
Y is H, unsubstituted or substituted lower alkyl, aryl, heteroaryl or unsubstituted or substituted cycloalkyl; and
Z is amino, mono- or di-substituted amino, halogen, alkyl, substituted alkyl, hydroxy, etherified or esterified hydroxy, nitro, cyano, carboxy, esterified carboxy, alkanoyl, carbamoyl, N-mono- or N,N-di-substituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkanesulfonyl or alkylphenylsulfonyl, and where, if more than one radical Z is present (m≧2), the substituents Z are identical or different;
or an N-oxide or a pharmaceutically acceptable salt thereof.
Additional RAF inhibitors include compounds as disclosed in PCT/EP2004/010688 which was filed Sep. 24, 2004, e.g compounds of formula V:

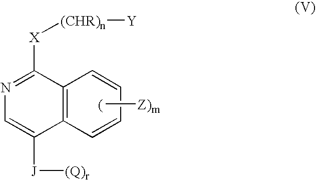

wherein
r is from 0-2;
n is from 0-2;
m is from 0-4;
J is aryl, heteroaryl, cycloalkyl or heterocycloalkyl, wherein
   aryl is an aromatic radical having from 6-14 carbon atoms, such as phenyl, naphthyl, fluorenyl and phenanthrenyl;
   heteroaryl is an aromatic radical having from 4-14, especially from 5-7 ring atoms, of which 1, 2 or 3 atoms are chosen independently from N, S and O, such as furyl, pyranyl, pyridyl, 1,2-, 1,3- and 1,4-pyrimidinyl, pyrazinyl, triazinyl, triazolyl, oxazolyl, quinazolyl, imidazolyl, pyrrolyl, isoxazolyl isothiazolyl, indolyl, isoindolinyl, quinolyl, isoquinolyl, purinyl, cinnolinyl, naphthyridinyl, phthalazinyl, isobenzofuranyl, chromenyl, purinyl, thianthrenyl, xanthenyl, acridinyl, carbazolyl and phenazinyl;
   cycloalkyl is a saturated cyclic radical having from 3-8, preferably from 5-6 ring atoms, such as cyclopropyl, cyclopentyl and cyclohexyl;
   heterocycloalkyl is a saturated cyclic radical having from 3-8, preferably from 5-6 ring atoms, of which 1, 2 or 3 atoms are chosen independently from N, S and O, such as piperidyl, piperazinyl, imidazolidinyl, pyrrolidinyl and pyrazolidinyl;
Q is a substituent on 1 or 2 carbon atoms selected from the group consisting of halogen, unsubstituted or substituted lower alkyl, —OR₂, —SR₂, —NR₂, —NRS(O)₂N(R)₂, —NRS(O)₂R, —S(O)R₂, —S(O)₂R₂, —OCOR₂, —C(O)R₂, —CO₂R₂, —NR—COR₂, —CON(R₂)₂, —S(O)₂N(R₂)₂, cyano, tri-methylsilanyl, unsubstituted or substituted aryl, unsubstituted or substituted heteroaryl, such as substituted or unsubstituted imidazolyl, and substituted or unsubstituted pyridinyl, unsubstituted or substituted cycloalkyl, unsubstituted or substituted heterocycloalkyl, such as substituted or unsubstituted piperidinyl, substituted or unsubstituted piperazolyl, substituted or unsubstituted tetrahydropyranyl, and substituted or unsubstituted azetidinyl, —C₁₋₄alkyl-aryl, —C₁₋₄alkyl-heteroaryl, —C₁₋₄alkyl-heterocyclyl, amino, mono- or di-substituted amino;
R is H or lower alkyl;
R₂ is unsubstituted or substituted alkyl, unsubstituted or substituted cycloalkyl, phenyl, —C₁₋₄alkyl-aryl, —C₁₋₄alkyl-heteroaryl or —C₁₋₄alkyl-heterocycloalkyl;
X is Y, —N(R)—, oxa, thio, sulfone, sulfoxide, sulfonamide, amide, or ureylene, preferably —NH—;
Y is H, lower alkyl, substituted or unsubstituted aryl, substituted or unsubstituted heteroaryl, substituted or unsubstituted cycloalkyl or substituted or unsubstituted heterocycloalkyl; and
Z is amino, mono- or di-substituted amino, halogen, alkyl, substituted alkyl, hydroxy, etherified or esterified hydroxy, nitro, cyano, carboxy, esterified carboxy, alkanoyl, carbamoyl, N-mono- or N,N-di-substituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkanesulfonyl or alkylphenylsulfonyl, and where, if more than one radical Z is present (m≧2), the substituents Z are identical or different;
or an N-oxide of the mentioned compound, wherein one or more N atoms carry an oxygen atom;
   or a pharmaceutically acceptable salt thereof.

In each case where citations of patent applications are given above, the subject matter relating to the compounds is hereby incorporated into the present application by reference. Comprised are likewise the pharmaceutically acceptable salts thereof, the corresponding racemates, diastereoisomers, enantiomers, tautomers, as well as the corresponding crystal modifications of above disclosed compounds where present, e.g. solvates, hydrates and polymorphs, which are disclosed therein. The compounds used as active ingredients in the combinations of the invention can be prepared and administered as described in the cited documents, respectively. Also within the scope of this invention is the combination of more than two separate active ingredients as set forth above, i.e., a pharmaceutical combination within the scope of this invention could include three active ingredients or more.

In accordance with the particular findings of the present invention, there is provided
1. A pharmaceutical combination comprising:
   a) a pyrimidylaminobenzamide compound of formula (I); and
   b) at least one RAF kinase inhibitor.
2. A method for treating or preventing proliferative disease in a subject in need thereof, comprising co-administration to said subject, e.g., concomitantly or in sequence, of a therapeutically effective amount of a pyrimidylaminobenzamide compound of formula (I) and a RAF kinase inhibitor, e.g., as disclosed above.
Examples of proliferative diseases include e.g. tumors, psoriasis, restenosis, sclerodermitis and fibrosis.
3. A pharmaceutical combination as defined under 1) above, e.g. for use in a method as defined under 2) above.
4. A pharmaceutical combination as defined under 1) above for use in the preparation of a medicament for use in a method as defined under 2) above.

Utility of the combination of the invention in a method as hereinabove specified, may be demonstrated in animal test methods as well as in clinic, for example in accordance with the methods hereinafter described.

A commercial package comprising a combination herein above or herein after described.

### A. Combined Treatment

Suitable clinical studies are, for example, open label, dose escalation studies in patients with proliferative diseases. Such studies prove in particular the synergism of the active ingredients of the combination of the invention. The beneficial effects on psoriasis or multiple sclerosis can be determined directly through the results of these studies which are known as such to a person skilled in the art. Such studies are, in particular, suitable to compare the effects of a monotherapy using the active ingredients and a combination of the invention. Preferably, the dose of agent (a) is escalated until the Maximum Tolerated Dosage is reached, and agent (b) is administered with a fixed dose. Alternatively, the agent (a) is administered in a fixed dose and the dose of agent (b) is escalated. Each patient receives doses of the agent (a) either daily or intermittent. The efficacy of the treatment can be determined in such studies, e.g., after 12, 18 or 24 weeks by evaluation of symptom scores every 6 weeks.

The administration of a pharmaceutical combination of the invention results not only in a beneficial effect, e.g. a synergistic therapeutic effect, e.g. with regard to alleviating, delaying progression of or inhibiting the symptoms, but also in further surprising beneficial effects, e.g. fewer side-effects, an improved quality of life or a decreased morbidity, compared with a monotherapy applying only one of the pharmaceutically active ingredients used in the combination of the invention.

A further benefit is that lower doses of the active ingredients of the combination of the invention can be used, for example, that the dosages need not only often be smaller but are also applied less frequently, which may diminish the incidence or severity of side-effects. This is in accordance with the desires and requirements of the patients to be treated.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

It is one objective of this invention to provide a pharmaceutical composition comprising a quantity, which is jointly therapeutically effective at targeting or preventing proliferative diseases a combination of the invention. In this composition, agent (a) and agent (b) may be administered together, one after the other or separately in one combined unit dosage form or in two separate unit dosage forms. The unit dosage form may also be a fixed combination.

The pharmaceutical compositions for separate administration of agent (a) and agent (b) or for the administration in a fixed combination, i.e. a single galenical composition comprising at least two combination partners (a) and (b), according to the invention may be prepared in a manner known per se and are those suitable for enteral, such as oral or rectal, and parenteral administration to mammals (warm-blooded animals), including humans, comprising a therapeutically effective amount of at least one pharmacologically active combination partner alone, e.g. as indicated above, or in combination with one or more pharmaceutically acceptable carriers or diluents, especially suitable for enteral or parenteral application.

Suitable pharmaceutical compositions contain, for example, from about 0.1% to about 99.9%, preferably from about 1% to about 60%, of the active ingredient(s). Pharmaceutical preparations for the combination therapy for enteral or parenteral administration are, for example, those in unit dosage forms, such as sugar-coated tablets, tablets, capsules or suppositories, or ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each of the combination partner of the combination of the invention may be administered simultaneously or sequentially and in any order, and the components may be administered separately or as a fixed combination. For example, the method of preventing or treating proliferative diseases according to the invention may comprise (i) administration of the first agent (a) in free or pharmaceutically acceptable salt form and (ii) administration of an agent (b) in free or pharmaceutically acceptable salt form, simultaneously or sequentially in any order, in jointly therapeutically effective amounts, preferably in synergistically effective amounts, e.g. in daily or intermittently dosages corresponding to the amounts described herein. The individual combination partners of the combination of the invention may be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of the combination partners employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration and the renal and hepatic function of the patient. A clinician or physician of ordinary skill can readily determine and prescribe the effective amount of the single active ingredients required to alleviate, counter or arrest the progress of the condition. Optimal precision in adhieving concentration of the active ingredients within the range that yields efficacy without toxicity requires a regimen based on the kinetics of the active ingredients' availability to target sites.

Daily dosages for agent (a) or (b) or will, of course, vary depending on a variety of factors, for example the compound chosen, the particular condition to be treated and the desired effect. In general, however, satisfactory results are achieved on administration of agent (a) at daily dosage rates of the order of ca. 0.03 to 5 mg/kg per day, particularly 0.1 to 5 mg/kg per day, e.g. 0.1 to 2.5 mg/kg per day, as a single dose or in divided doses. Agent (a) and agent (b) may be administered by any conventional route, in particular enterally, e.g. orally, e.g. in the form of tablets, capsules, drink solutions or parenterally, e.g. in the form of injectable solutions or suspensions. Suitable unit dosage forms for oral administration comprise from ca. 0.02 to 50 mg active ingredient, usually 0.1 to 30 mg, e.g. agent (a) or (b), together with one or more pharmaceutically acceptable diluents or carriers therefore.

Agent (b) may be administered to a human in a daily dosage range of 0.5 to 1000 mg. Suitable unit dosage forms for oral administration comprise from ca. 0.1 to 500 mg active ingredient, together with one or more pharmaceutically acceptable diluents or carriers therefore.

The administration of a pharmaceutical combination of the invention results not only in a beneficial effect, e.g. a synergistic therapeutic effect, e.g. with regard to inhibiting the unregulated proliferation of haematological stem cells or slowing down the progression of leukemias, such as CML or AML, or the growth of tumors, but also in further surprising beneficial effects, e.g. less side-effects, an improved quality of life or a decreased morbidity, compared to a monotherapy applying only one of the pharmaceutically active ingredients used in the combination of the invention.

A further benefit is that lower doses of the active ingredients of the combination of the invention can be used, for example, that the dosages need not only often be smaller but are also applied less frequently, or can be used in order to diminish the incidence of side-effects. This is in accordance with the desires and requirements of the patients to be treated.

B. Diseases to be treated

The term “proliferative disease” includes but is not restricted to tumors, psoriasis, restenosis, sclerodermitis and fibrosis.

The term haematological malignancy, refers in particular to leukemias, especially those expressing Bcr-Abl, c-Kit or Flt-3, and includes, but is not limited to, chronic myelogenous leukemia and acute lymphocyte leukemia (ALL), especially the Philadelphia chromosome positive acute lymphocyte leukemia (Ph+ALL) as well as ST1571-resistant leukemia.

The term “a solid tumor disease” especially means ovarian cancer, breast cancer, cancer of the colon and generally the gastrointestinal tract, cervix cancer, lung cancer, e.g. small-cell lung cancer and non-small-cell lung cancer, head and neck cancer, bladder cancer, cancer of the prostate or Kaposi's sarcoma.

The combinations according to the invention, that inhibit the protein kinase activities mentioned, especially tyrosine protein kinases mentioned above and below, can therefore be used in the treatment of protein kinase dependent diseases. Protein kinase dependent diseases are especially proliferative diseases, preferably benign or especially malignant tumours (for example carcinoma of the kidneys, liver, adrenal glands, bladder, breast, stomach, ovaries, colon, rectum, prostate, pancreas, lungs, vagina or thyroid, sarcoma, glioblastomas and numerous tumours of the neck and head, as well as leukemias). They are able to bring about the regression of tumours and to prevent the formation of tumour meta-stases and the growth of (also micro)metastases. In addition they can be used in epidermal hyperproliferation (e.g. psoriasis), in prostate hyperplasia, and in the treatment of neo-plasias, especially of epithelial character, for example mammary carcinoma. It is also possible to use the combinations of the present invention in the treatment of diseases of the immune system insofar as several or, especially, individual tyrosine protein kinases are involved; furthermore, the combinations of the present invention can be used also in the treatment of diseases of the central or peripheral nervous system where signal transmission by at least one tyrosine protein kinase, especially selected from those mentioned specifically, is involved.

In chronic myelogeous leukemia (CML), a reciprocally balanced chromosomal translocation in hematopoietic stem cells (HSCs) produces the BCR-ABL hybrid gene. The latter encodes the oncogenic Bcr-Abl fusion protein. Whereas ABL encodes a tightly regulated protein tyro-sine kinase, which plays a fundamental role in regulating cell proliferation, adherence and apoptosis, the BCR-ABL fusion gene encodes as constitutively activated kinase, which trans-forms HSCs to produce a phenotype exhibiting deregulated clonal proliferation, reduced capacity to adhere to, the bone marrow stroma and a reduces apoptotic response to mutagenic stimuli, which enable it to accumulate progressively more malignant transformations. The re-sulting granulocytes fail to develop into mature lymphocytes and are released into the circu-lation, leading to a deficiency in the mature cells and increased susceptibility to infection. ATP-competitive inhibitors of Bcr-Abl have been described which prevent the kinase from activating mitogenic and anti-apoptotic pathways (e.g. P-3 kinase and STAT5), leading to the death of the BCR-ABL phenotype cells and thereby providing an effective therapy against CML. The combinations of the present invention are thus especially appropriate for the therapy of diseases related to its overexpression, especially leukemias, such as leukemias, e.g. CML or ALL.

The RAF kinase inhibiting property of the combinations of the present invention makes them useful as therapeutic agents for the treatment for proliferative diseases characterized by an aberrant MAP kinase signaling pathway, particularly many cancers characterized by overexpression of RAF kinase or an activating mutation of RAF kinase, such as melanoma having mutated B-RAF, especially wherein the mutated B-RAF is the V599E mutant. The present invention also provides a method of treating other conditions characterized by an aberrant MAP kinase signaling pathway, particularly where B-RAF is mutated, for example benign Nevi moles having mutated B-RAF, with the combinations of the present invention.

In general, the disease characterized by excessive signaling through the MAP kinase signaling pathway is a proliferative disease, particularly a cancer characterized by increased RAF kinase activity, for example one which overexpresses wild-type B- or C-RAF kinase, or that expresses an activating mutant RAF kinase, for example a mutant B-RAF kinase. Cancers wherein a mutated RAF kinase has been detected include melanoma, colorectal cancer, ovarian cancer, gliomas, adenocarcinomas, sarcomas, breast cancer and liver cancer. Mutated B-RAF kinase is especially prevalent in many melanomas.

In accordance with the present invention, a sample of diseased tissue is taken from the patient, for example, as a result of a biopsy or resection, and tested to determine whether the tissue produces a mutant RAF kinase, such as a mutant B-RAF kinase or overexpresses a wild-type RAF kinase, such as wild-type B- or C-RAF kinase. If the test indicates that mutant RAF kinase is produced or that a RAF kinase is overproduced in the diseased tissue, the patient is treated by administration of an effective RAF-inhibiting amount of a RAF inhibitor compound described herein.

Further in accordance with the invention is the use of combinations of the present invention described herein for the preparation of a medicament for the treatment of melanoma which comprises (a) testing melanoma tissue from the patient to determine whether the melanoma tissue expresses mutant RAF kinase or overexpresses a wild-type RAF kinase and (b) treating the patient if the melanoma tissue is found to overexpress a wild type RAF kinase or express an activating mutant B-RAF kinase with an effective RAF kinase inhibiting amount of combinations of the present invention.

However, it is also possible to down-regulate the MAP kinase signaling pathway with a RAF kinase inhibiting compound If another kinase in the cascade is the cause of excessive signaling in the pathway. Thus, the present invention further relates to the treatment of a disease characterized by excessive signaling in the MAP kinase signaling pathway attributed to a cause other than an activating mutation in or overexpression of a RAF kinase.

The combinations of the present invention primarily inhibit the growth of blood vessels and are thus, for example, effective against a number of diseases associated with deregulated angiogenesis, especially diseases caused by ocular neovascularisation, especially retinopathies, such as diabetic retinopathy or age-related macula degeneration, psoriasis, haemangioblastoma, such as haemangioma, mesangial cell proliferative disorders, such as chronic or acute renal diseases, e.g. diabetic nephropathy, malignant nephrosclerosis, thrombotic microangiopathy syndromes or transplant rejection, or especially inflammatory renal disease, such as glomerulonephritis, especially mesangioproliferative glomerulonephritis, haemolytic-uraemic syndrome, diabetic nephropathy, hypertensive nephrosclerosis, atheroma, arterial restenosis, autoimmune diseases, diabetes, endometriosis, chronic asthma, and especially neoplastic diseases (solid tumors, but also leukemias and other haematological malignancies), such as especially breast cancer, cancer of the colon, lung cancer (especially small-cell lung cancer), cancer of the prostate or Kaposi's sarcoma. Combinations of the present invention inhibit the growth of tumours and are especially suited to preventing the metastatic spread of tumors and the growth of micrometastases.

The invention relates to a method of treating myeloma, especially myeloma which is resistant to conventional chemotherapy. The term “myeloma” as used herein relates to a tumor composed of cells of the type normally found in the bone marrow. The term “multiple myeloma” as used herein means a disseminated malignant neoplasm of plasma cells which is characterized by multiple bone marrow tumor foci and secretion of an M component (a monoclonal immunoglobulin fragment), associated with widespread osteolytic lesions resulting in bone pain, pathologic fractures, hypercalcaemia and normochromic normocytic anaemia. Multiple myeloma is incurable by the use of conventional and high dose chemotherapies. Tthe invention relates to a method of treating myeloma, especially myeloma which is resistant to conventional chemotherapy.

1. A pharmaceutical combination comprising:
a) a pyrimidylaminobenzamide compound of formula (I), and
b) at least one RAF kinase inhibitor, wherein formula (I) is: 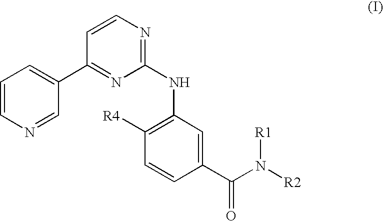
wherein
R₁ represents hydrogen, lower alkyl, lower alkoxy-lower alkyl, acyloxy-lower alkyl, carboxy-lower alkyl, lower alkoxycarbonyl-lower alkyl, or phenyl-lower alkyl:
R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted:
and R₃ represents hydroxy, lower alkoxy, acyloxy, carboxy, lower alkoxycarbonyl, carbamoyl, N-mono- or N,N-disubstituted carbamoyl, amino, mono- or disubstituted amino, cycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted;
or wherein R₁ and R₂ together represent alkylene with four, five or six carbon atoms optionally mono- or disubstituted by lower alkyl, cycloalkyl, heterocyclyl, phenyl, hydroxy, lower alkoxy, amino, mono- or disubstituted amino, oxo, pyridyl, pyrazinyl or pyrimidinyl; benzalkylene with four or five carbon atoms: oxaalkylene with one oxygen and three or four carbon atoms; or azaalkylene with one nitrogen and three or four carbon atoms wherein nitrogen is unsubstituted or substituted by lower alkyl, phenyl-lower alkyl, lower alkoxycarbonyl-lower alkyl, carboxy-lower alkyl, carbamoyl-lower alkyl, N-mono- or N,N-disubstituted carbamoyl-lower alkyl, cycloalkyl, lower alkoxycarbonyl, carboxy, phenyl, substituted phenyl, pyridinyl, pyrimidinyl, or pyrazinyl:
R₄ represents hydrogen, lower alkyl, or halogen.

2. A method for treating or preventing a proliferative disease in a subject in need thereof, comprising co-administration to said subject, e.g. concomitantly or in sequence, of a therapeutically effective amount of at least one RAF kinase inhibitor and a pyrimidylaminobenzamide compound of formula (I).

3. A pharmaceutical combination according to claim 1.

4. A pharmaceutical combination according to claim 1 for use in the preparation of a medicament for the treatment of a proliferative disease.

5. A pharmaceutical combination according to claim 1 wherein agent a) is selected from 4-Methyl-3-[[4-(3-pyridinyl)-2-pyrimidinyl]amino]-N-[5-(4-methyl-1H-imidazol-1-yl)-3-(trifluoromethyl)phenyl]benzamide.

6. A method of claim 2 wherein the proliferative disease is myeloma.

7. A method of claim 2, wherein pyrimidylaminobenzamide compound of formula (I) is 4-Methyl-3-[[4-(3-pyridinyl)-2-pyrimidinyl]amino]-N-[5-(4-methyl-1H-imidazol-1-yl)-3-(trifluoromethyl)phenyl]benzamide.

8. A method of claim 2, comprising administering a combination of a RAF inhibitor and a pyrimidylaminobenzamide compound of formula (I), wherein the RAF inhibitor is selected from (4-tert-butyl-phenyl)-(4-pyridin-4-ylmethyl-isoquinolin-1-yl)-amine; [4,7′]biisoquinolinyl-1-yl-4-(tert-butyl-phenyl)-amine; (4-tert-Butyl-phenyl)-(4-quinazolin-6-yl-isoquinolin-1-yl)-amine; [4,7′]Biisoquinolinyl-1-yl-(2-tert-butyl-pyrimidin-5-yl)-amine and combinations thereof.

9. A combination according to claim 1 wherein the RAF inhibitor is selected from (4-tert-butyl-phenyl)-(4-pyridin-4-ylmethyl-isoquinolin-1-yl)-amine; [4,7′]biisoquinolinyl-1-yl-4-(tert-butyl-phenyl)-amine; (4-tert-Butyl-phenyl)-(4-quinazolin-6-yl-isoquinolin-1-yl)-amine; [4,7′]Biisoquinolinyl-1-yl-(2-tert-butyl-pyrimidin-5-yl)-amine and combinations thereof.

10. A commercial package comprising a combination according to claim 1.
